(19) United States
(12) Patent Application Publication
(10) Pub. No.: US 2006/0287656 A1
(43) Pub. Date: Dec. 21, 2006

(54) Medical instrument for removing objects from narrow passages

(76) Inventors: Stuart I. Brown, St. Andrews (GB); Timothy Graham Frank, Wormit Newport-On-Tay (GB); Leslie Kelly, Cupar (GB); Rodney Mountain, Brunton (GB); Ian Rutherford, Dundee (GB)

Correspondence Address: ST. ONGE STEWARD JOHNSTON & REENS, LLC, 986 BEDFORD STREET, STAMFORD, CT, 06905-5619, US

(21) Appl. No.: 11/450,609

(22) Filed: Jun. 9, 2006

(30) Foreign Application Priority Data
Jun. 9, 2005 (DE) 10 2005 026 466.2

(51) Int. Cl. A61B 17/22

(52) U.S. Cl. 606/127

(57) ABSTRACT

The invention relates to a medical instrument for removing objects from narrow passages, which instrument has a shaft on whose distal end a hook-shaped curvature is configured. In order to produce a medical instrument that is easy to operate and ensures reliable removal of objects even in cramped space conditions, it is proposed with the invention that the distal end of the shaft can be converted between an essentially linear form and a form that has a hook-shaped curvature.

### FIELD OF THE INVENTION

The invention relates to a medical instrument for removing objects from narrow passages, which instrument includes a shaft on whose distal end a hook-shaped curvature is configured.

### BACKGROUND OF THE INVENTION

Hook-shaped medical instruments of this type are known in the art, for instance, from ear, nose and throat medicine, and are used to remove cerumen, the so-called earwax, or other foreign objects from the outer auditory canal. The disadvantage of these known instruments is that it is difficult to get the hook-shaped distal end of the shaft placed behind the object that is to be removed in order then to pull the object out of the auditory canal by means of the hook. Depending on the prevailing space conditions, there is a constant danger in practice that in introducing the hook-shaped shaft, the object that is to be removed is pressed farther into the auditory canal.

Consequently, it is the object of this invention to produce a medical instrument for removing objects from narrow passages, which instrument is easy to operate and ensures dependable removal of objects even in narrow spaces.

### SUMMARY OF THE INVENTION

This object is fulfilled by the invention in a manner characterized by the fact that the distal end of the shaft can be converted between an essentially linear shape and a shape having the hook-shaped curvature.

Owing to the design of the shaft according to the invention, in which its distal end can be converted between a linear and a hook-shaped form, it is possible for the first time to introduce the shaft first in a non-curved form into the passage to be examined and, only after it is inside the passage and behind an object that is to be removed, to switch the shaft into the curved hook shape. Because of this convertibility of the distal end of the shaft according to the invention, with an instrument configured in this way there is no danger that the object that is to be removed from the passage is pushed farther into the passage when the shaft is introduced into the passage.

According to a practical embodiment of the invention it is proposed that the shaft can be introduced into a passage in an essentially linear form by means of a guide, and that the shaft outside the guide can be converted into the form that has the hook-shaped curvature. The use of the guide allows an exact positioning of the shaft inside the passage.

According to the invention the distal end of the shaft is advantageously converted into the hook-shaped curved form independently, owing to the use of a shaft consisting of a pre-bent material.

Particularly appropriate materials for configuring the shaft in accordance with the invention are so-called superelastic working materials such as NiTi alloys, which possess the necessary elasticity and stiffness in order, first, to be flexed in an essentially linear form and, second, thanks to the memory effect, to assume the pre-bent hook form again, so that they are sufficiently stable in curved form to be able to extract from the passage the object that is to be removed, even against a certain resistance.

According to a first embodiment of the invention it is proposed that the guide for introducing the shaft into the passage is configured as a cylindrical piston system, where the shaft is positioned inside the cylinder so that it can be pushed by the piston. This guide configured in the manner of a hypodermic needle is easy to operate and allows precise aim in inserting the shaft. The sliding of the shaft by the piston can, according to the invention, be facilitated if the proximal end of the piston is configured as a thumb support.

To prevent injuries to the passage that is to be examined, it is further proposed with the invention that the cylinder is pre-tensioned by means of a spring element into a position pulling the shaft into the cylinder. As soon as the operator of the inventive medical instrument no longer exerts pressure on the cylinder, the shaft is thus again drawn into the cylinder by means of the spring element and the cylinder.

With a second practical embodiment of the invention it is proposed that the guide is configured as an essentially cylindrical-shaped handle, which on the distal side has a hollow shaft for supportive guidance of the shaft. The hollow shaft is advantageously configured in an arched shape to allow the operator a better view into the passage that is to be examined.

According to the invention, displacement of the shaft in this embodiment occurs advantageously by means of an axially movable slide block that is positioned in the handle and in turn is actively connected with a bolt mounted on the exterior of the handle.

To prevent, even with this embodiment, any injuries of the passage that is to be examined, it is further proposed with the invention that the slide block is pre-tensioned by means of a spring element into a position that draws the shaft into the hollow shaft. As soon as the operator of the medical instrument according to this invention no longer exerts pressure on the slide block by means of the bolt, the shaft is thus drawn back into the hollow shaft by means of the spring element and the slide block.

According to an alternative embodiment of the invention, it is proposed that the guide for inserting the shaft into the passage is configured as a narrow guide tube.

It is further proposed with the invention that the free distal end of the shaft is configured as blunted, for instance in the shape of a loop, to exclude the possibility of causing an injury of the passage to be examined by means of the free end of the shaft that is inserted at first in linear form into the passage.

It is finally proposed with the invention that the shaft or the guide combined with the shaft is introduced into the passage to be examined by means of a speculum. These funnel-shaped instruments are well known, for instance, from the ear, nose, and throat sector for their ability to be inserted into body cavities, for instance the outer auditory canal. When examination instruments are inserted, the speculum prevents injury to the passage that is to be examined.

Additional characteristics and advantages of the invention can be seen from the appended illustrations, depicting in merely schematic form two embodiments of a medical instrument according to the invention for removing objects from narrow passages.

### BRIEF DESCRIPTION OF THE DRAWINGS

FIG. 1a shows a longitudinal section through a first embodiment of a medical instrument according to the invention, with inserted shaft.

FIG. 1b shows a view as in FIG. 1 but showing the shaft in the opened position.

FIG. 2a shows an overhead view of a second embodiment of a medical instrument according to the invention.

FIG. 2b shows a section along the line IIb-IIb according to FIG. 2a.

FIG. 3a shows a schematic view of the use of the medical instrument according to the invention that is seen in FIGS. 1a and 1b during the insertion.

FIG. 3b shows a view according to FIG. 3a but showing the working position.

FIG. 4 shows a schematic side view of a speculum.

### DETAILED DESCRIPTION OF THE INVENTION

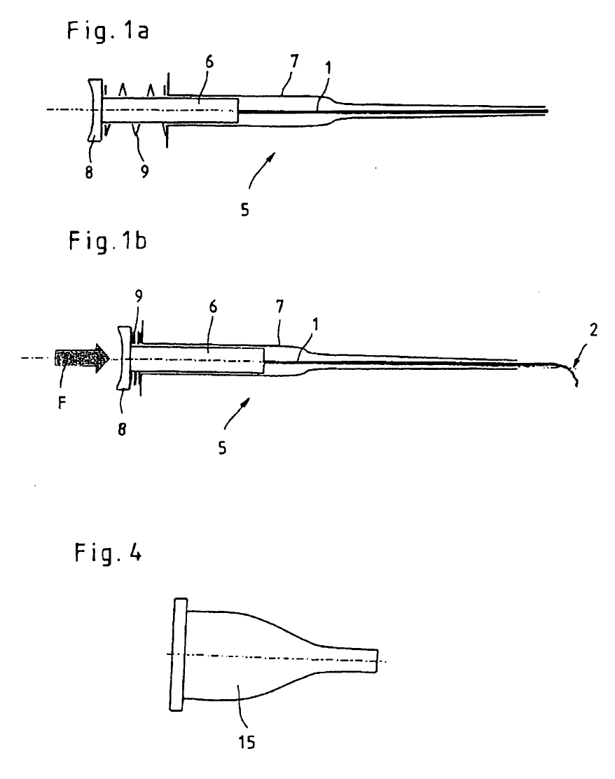

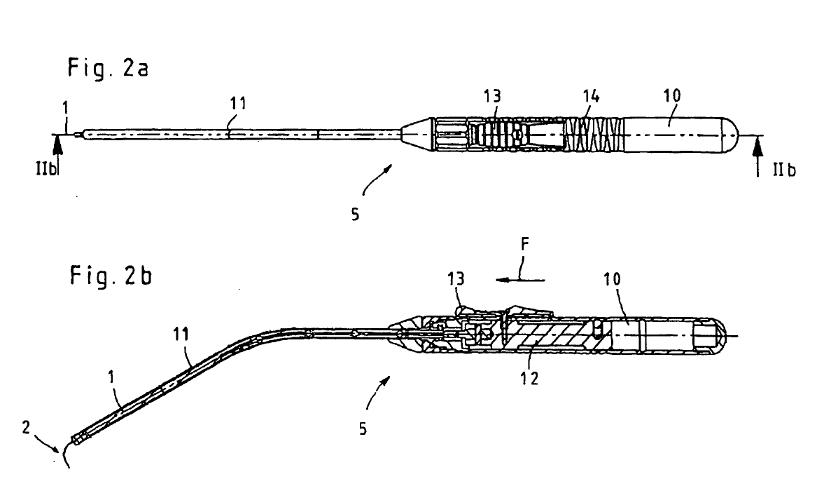

The medical instrument shown in FIGS. 1a to 2b for removing objects from narrow passages consists essentially of a wire-shaped shaft 1, on whose distal end a hook-shaped curvature 2 can be configured for reaching behind a foreign body positioned in a passage 3.

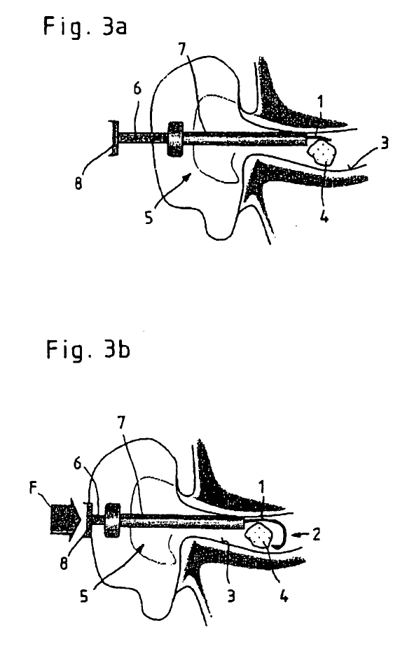

The shaft 1 is introduced into the passage 3, for instance the outer auditory canal of an ear, in practice by means of a guide 5, which is configured as a cylinder-piston system in the shape of a hypodermic needle in the first embodiment illustrated in FIGS. 1a, 1b, and FIGS. 3a and 3b. As can be seen from FIGS. 1a and 1b, the proximal end of the shaft 1 is connected with the distal end of a piston 6 of the cylinder-piston system, while the remaining part of the shaft 1 is positioned in a cannular-shaped cylinder 7 of the cylinder-piston system.

To configure the hook-shaped curvature 2 on the distal end of the shaft 1, the shaft 1 consists of an elastic material, such as a super-elastic NiTi alloy, which is pre-bent in a hook-shaped form. The elasticity of the material, however, allows the shaft to be converted into an essentially linear form in order to be able to introduce it by means of the guide 5 into the passage 3 that is to be examined. As soon as the shaft 1 again leaves the guide 5, it independently assumes the pre-bent hook-shaped form again.

To ensure safe operation of the guide 5, configured as a cylinder-piston system, the proximal end of the piston 6 is configured as a thumb support 8, by which the operator of the medical instrument can exert pressure with his thumb in the direction of the arrow F on the piston 6, in order to press the shaft 1 out of the cylinder 7. The piston 6 is pressed into the cylinder 7 against the force of a spring element 9 configured as a pressure spring, by which the piston 6 is pre-tensioned into a position drawing the shaft 1 into the cylinder 7. This spring element 9 ensures that the shaft 1 is automatically drawn back into the cylinder 7 of the guide 5 as soon as the operator of the medical instrument no longer exerts pressure force F on the piston 6. Thus, any accidental injury of the passage 3 by failure to use the guide 5 can be ruled out.

In the second embodiment, depicted in FIGS. 2a and 2b, the guide 5 for the shaft 1 consists of an essentially cylindrical handle 10, which on the distal side has a preferably arched hollow shaft 11 for supported guidance of the shaft 1. In addition to the illustrated arched configuration of the hollow shaft 11, it is also possible of course to configure the hollow shaft 11 as straight.

In this embodiment the shaft 1 is converted between the starting position inserted in the hollow shaft 11 and the opened working position illustrated in FIGS. 2a and 2b by means of a slide block 12 that is mounted so that it can be pushed axially in the handle 10 and that is in active connection with a bolt 13 positioned on the exterior of the handle 10.

As can be seen from FIG. 2b, the proximal end of the shaft 1 is connected with the distal end of a slide block 12, while on the proximal end of the slide block 12 a spring element 14 configured as a tension spring engages, by means of which the slide block 12 is pre-tensioned into a position drawing the shaft 1 into the hollow shaft 11. This spring element 14 ensures that the shaft 1 is automatically drawn back into the hollow shaft 11 of the guide 5, as soon as the operator of the medical instrument no longer exerts pressure force F on the slide block 12 by means of the bolt 13.

Operation of the above-described medical instrument for removing objects 4 from a narrow passage 3 is described hereafter on the basis of the embodiment of FIGS. 1a and 1b.

FIGS. 3a and 3b are schematic views of the practical use of the previously described medical instrument from the example of a foreign body 4 that is to be removed from the outer auditory canal 3 of an ear, and which can be for instance hardened cerumen, so-called earwax.

To prevent injuries to the auditory canal 3 and to facilitate the insertion of the guide 5 into the auditory canal 3, first a funnel-shaped speculum 15 is inserted into the auditory canal 3. The speculum illustrated in FIG. 4 was omitted from FIGS. 3a and 3b for reasons of better clarity. Likewise, additional medical instruments such as an otoscope for visual monitoring of the operation, were not shown.

The guide 5 is introduced into the auditory canal 3 by means of the speculum 15 until the distal end of the cylinder 7 has almost reached the foreign body 4 that is to be removed. Then the operator of the medical instrument, by means of the thumb support 8, presses the piston 6 into the cylinder 7 of the guide 5, so that the shaft is pushed out of the distal end of the cylinder 7 as shown in FIG. 3a.

The shaft extending out of the cylinder 7 expels the foreign body 4 that is to be removed and, on further pushing of the shaft 1 out of the cylinder 7, because of the pre-tensioning and the memory effect of the material behind the foreign body 4, forms the pre-formed curvature 2 independently, as can be seen in FIG. 3b.

To remove the foreign body 4 from the auditory canal 3, the operator of the medical instrument, with the piston 6 continuously pressed in, needs only to pull the guide 5 outward from the auditory canal 3. The curvature 2, which reaches behind the foreign body 4, apprehends said foreign body and removes it from the auditory canal 3.

In addition to the selection of an appropriately elastic material for configuring the shaft 1, to allow repeated extending and re-bending of the distal end of the shaft 1, the material should be selected, in terms of material stiffness and/or material strength, in such a way that the curvature 2 that reaches behind the foreign body 4 that is to be removed does not bend back immediately if a resistance pressure is exerted on the hook-shaped curvature 2 by the foreign body 4.

A medical instrument of this configuration for removing objects 4 from narrow passages 3 is distinguished in that it is of simple construction, is easy to operate, and ensures a reliable removal of foreign bodies 4 from the passage 3 that is to be examined. Because the shaft 1 can be inserted into the passage 3 by the guide 5 in extended form, that is without curvature 2, and the curvature 2 serving to grasp the foreign body 4 is configured only behind the foreign body 4, there is no danger that the foreign body 4 becomes pushed farther into the passage 3 when the shaft 1 is inserted into the passage 3.

1. A medical instrument for removing objects from narrow passages, which instrument has a shaft on whose distal end a hook-shaped curvature is configured, distinguished in that the distal end of the shaft can be converted between an essentially linear form and a form that has the hook-shaped curvature.

2. A medical instrument according to claim 1, distinguished in that the shaft can be inserted in an essentially linear form by means of a guide into a passage and that the shaft can be converted outside the guide into the form that has the hook-shaped curvature.

3. A medical instrument according to claim 1, distinguished in that the distal end of the shaft independently assumes the form that includes the hook-shaped curvature.

4. A medical instrument according to claim 1, distinguished in that the shaft consists of a pre-bent elastic material.

5. A medical instrument according to claim 4, distinguished in that the shaft consists of an elastic material, in particular a NiTi alloy.

6. A medical instrument according to claim 2, distinguished in that the guide is configured as a narrow guide tube.

7. A medical instrument according to claim 2, distinguished in that the guide is configured as a cylinder-piston system, where the shaft is mounted so that it can slide in the cylinder by means of the piston.

8. A medical instrument according to claim 7, distinguished in that the piston is pre-tensioned into a position that draws the shaft into the cylinder.

9. A medical instrument according to claim 7, distinguished in that the proximal end of the piston is configured as a thumb support.

10. A medical instrument according to claim 2, distinguished in that the guide is configured as an essentially cylindrical handle, which on the distal side has a hollow shaft for supportive guidance of the shaft.

11. A medical instrument according to claim 10, distinguished in that the shaft can be driven by means of a slide block mounted so that it can slide axially in the handle.

12. A medical instrument according to claim 11, distinguished in that the slide block can be activated by a bolt positioned on the exterior of the handle.

13. A medical instrument according to claim 11, distinguished in that the slide block is pre-tensioned by a spring element into a position drawing the shaft into the hollow shaft.

14. A medical instrument according to claim 1, distinguished in that the free distal end of the shaft is configured as blunted.

15. A medical instrument according to claim 1, distinguished in that the shaft can be inserted into the passage by means of a speculum.
